(19) United States
(12) Patent Application Publication
(10) Pub. No.: US 2009/0306380 A1
(43) Pub. Date: Dec. 10, 2009

(54) Process for preparing 4-amino-2-alkylthio-5-pyrimidinecarbaldehyde

(76) Inventors: Shigeyoshi Nishino, Yamaguchi (JP); Shoji Shikita, Yamaguchi (JP); Tadashi Murakami, Yamaguchi (JP)

Correspondence Address: BIRCH STEWART KOLASCH & BIRCH, PO BOX 747, FALLS CHURCH, VA, 22040-0747, US

(21) Appl. No.: 11/919,179

(22) PCT Filed: Apr. 24, 2006
(86) PCT No.: PCT/JP2006/308507

§ 371 Date: Oct. 24, 2007

(30) Foreign Application Priority Data
Apr. 25, 2005 (JP) 2005-126090
Jun. 7, 2005 (JP) 2005-166512
Jun. 7, 2005 (JP) 2005-166513

(51) Int. Cl. C07D 239/47; C07C 255/07

(52) U.S. Cl. 544/317; 558/379

(57) ABSTRACT

Provided are a process for preparing a 4-amino-2-alkylthio-5-pyrimidinecarbaldehyde, which is industrially advantageous in that a 4-amino-2-alkylthio-5-pyrimidinecarbaldehyde can be prepared in high yield in a simple way, an intermediate used in the process, and a process for preparing the intermediate, which is industrially advantageous in that the intermediate can be prepared safely in high yield with ease.

The present invention is directed to: a process for preparing an alkali metal salt of 3,3-dialkoxy-2-hydroxymethylenepropanenitrile, comprising reacting at least one nitrile compound selected from the group consisting of a 3,3-dialkoxypropanenitrile and a 3-alkoxy-2-propenenitrile, with a formic acid ester at −10 to 30° C. in the presence of a base comprising an alkali metal; an alkali metal salt of 4-amino-2-mercapto-5-pyrimidinecarbaldehyde; a process for preparing an alkali metal salt of 4-amino-2-mercapto-5-pyrimidinecarbaldehyde, comprising reacting an alkali metal salt of 3,3-dialkoxy-2-hydroxymethylenepropanenitrile with thiourea; a process for preparing a 4-amino-2-alkylthio-5-pyrimidinecarbaldehyde, comprising reacting an alkali metal salt of 4-amino-2-mercapto-5-pyrimidinecarbaldehyde with an alkylating agent; and an use of an alkali metal salt of 4-amino-2-mercapto-5-pyrimidinecarbaldehyde in the preparation of a 4-amino-2-alkylthio-5-pyrimidinecarbaldehyde.

### FIELD OF THE INVENTION

The present invention relates to a process for preparing a 4-amino-2-alkylthio-5-pyrimidinecarbaldehyde, an alkali metal salt of 4-amino-2-mercapto-5-pyrimidinecarbaldehyde as an intermediate used in the preparation of a 4-amino-2-alkylthio-5-pyrimidinecarbaldehyde, and a process for preparing the same. The 4-amino-2-alkylthio-5-pyrimidinecarbaldehyde is a useful compound as a starting material or a synthetic intermediate for pharmaceuticals or pesticides.

### BACKGROUND ART

Heretofore, as a process for preparing a 4-amino-2-alkylthio-5-pyrimidinecarbaldehyde, for example, a process for preparing 4-amino-2-methylthio-5-pyrimidinecarbaldehyde by reacting 4-amino-2-mercapto-5-pyrimidinecarbaldehyde and methyl iodide with potassium carbonate has been disclosed (see, for example, patent document 1). However, this process involves problems that excess methyl iodide must be used and it takes a long time until the reaction is completed. In this process, a starting material, 4-amino-2-mercapto-5-pyrimidinecarbaldehyde which is synthesized from a potassium salt of 3,3-diethoxy-2-formylpropionitrile and thiourea is formed as a thick slurry (see, for example, patent document 1) and hence, the filtering properties of the slurry are too poor to isolate for using it as a starting material. Therefore, the development of a suitable starting material for the preparation of a 4-amino-2-alkylthio-5-pyrimidinecarbaldehyde has been also desired.

As a process for preparing an alkali metal salt of 3,3-dialkoxy-2-hydroxymethylenepropanenitrile, such as a potassium salt of 3,3-diethoxy-2-formylpropionitrile which is used as a starting material in the above process, for example, a process by reacting 3,3-dimethoxypropanenitrile or 3-methoxy-2-propenenitrile and methyl formate at 40 to 100° C. with sodium methoxide (see, for example, patent document 2), and a process in which 3,3-diethoxypropanenitrile and methyl formate are reacted with potassium t-butoxide has been disclosed (see, for example, patent document 1). However, these processes disadvantageously generate a great amount of carbon monoxide which is toxic gas, and are undesirable as an industrial preparing process, so that, there has been also desired an industrially suitable process for preparing an alkali metal salt of 3,3-dialkoxy-2-hydroxymethylenepropanenitrile which can prepare the alkali metal salt safely in high yield.

Patent document 1: Japanese Unexamined Patent Publication (kohyo) No. 2004-507540
Patent document 2: Japanese Unexamined Patent Publication No. Sho 60-19755

### DISCLOSURE OF THE INVENTION

### Problems to be Solved by the Invention

An object of the present invention is to solve the above problems, and to provide an industrially suitable process for preparing a 4-amino-2-alkylthio-5-pyrimidinecarbaldehyde which can prepare a 4-amino-2-alkylthio-5-pyrimidinecarbaldehyde simply in high yield from the optimum starting material, an alkali metal salt of 4-amino-2-mercapto-5-pyrimidinecarbaldehyde as an intermediate used in this process, and an industrially suitable process for preparing the intermediate which can prepare the intermediate safely in high yield with ease.

### Means to Solve the Problems

The present invention is directed to a process for preparing an alkali metal salt of 3,3-dialkoxy-2-hydroxymethylenepropanenitrile {hereinafter, referred to as “compound (4)”} represented by the following general formula (4):

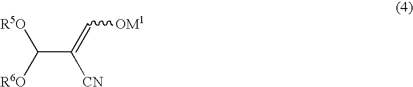

wherein each of R⁵ and R⁶ which may be the same or different represents an alkyl group, and M¹ represents an alkali metal atom,

wherein the process comprises reacting at least one nitrile compound selected from the group consisting of a 3,3-dialkoxypropanenitrile {hereinafter, referred to as “compound (1)”} represented by the following general formula (1):

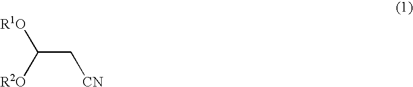

wherein each of R¹ and R² which may be the same or different represents an alkyl group
   and a 3-alkoxy-2-propenenitrile {hereinafter, referred to as “compound (2)”} represented by the following general formula (2):

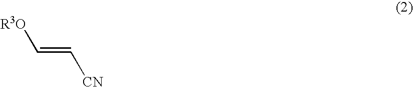

wherein R³ represents an alkyl group,
   with a formic acid ester {hereinafter, referred to as “compound (3)”} represented by the following general formula (3):

HCO₂R⁴ (3)
wherein R⁴ represents an alkyl group,
excluding a methyl group,
   at −10 to 30° C. in the presence of a base comprising an alkali metal.

The present invention is also directed to an alkali metal salt of 4-amino-2-mercapto-5-pyrimidinecarbaldehyde {hereinafter, referred to as “compound (5)”} represented by the following general formula (5):

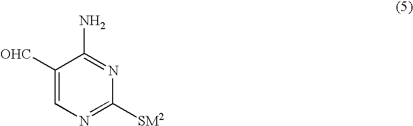

wherein M² represents an alkali metal atom.

The present invention is also directed to a process for preparing an alkali metal salt of 4-amino-2-mercapto-5-pyrimidinecarbaldehyde {compound (5)} represented by the following general formula (5):

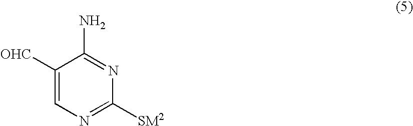

wherein M² represents an alkali metal atom,

wherein the process comprises reacting an alkali metal salt of 3,3-dialkoxy-2-hydroxymethylenepropanenitrile {compound (4)} represented by the following general formula (4):

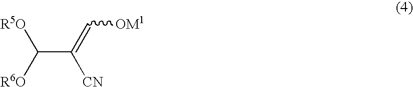

wherein each of R⁵ and R⁶ which may be the same or different represents an alkyl group, and M¹ represents an alkali metal atom
   with thiourea.

The present invention is also directed to a process for preparing a 4-amino-2-alkylthio-5-pyrimidinecarbaldehyde {hereinafter, referred to as “compound (6)”} represented by the following general formula (6):

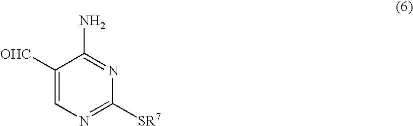

wherein R⁷ represents an alkyl group,

wherein the process comprises reacting an alkali metal salt of 4-amino-2-mercapto-5-pyrimidinecarbaldehyde {compound (5)} represented by the following general formula (5):

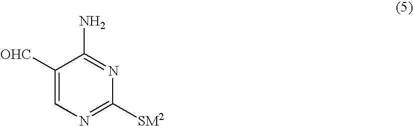

wherein M² represents an alkali metal atom
   with an alkylating agent.

Further, the present invention is directed to the use of an alkali metal salt of 4-amino-2-mercapto-5-pyrimidinecarbaldehyde represented by the following general formula (5):

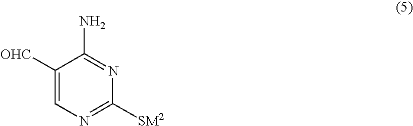

wherein M² represents an alkali metal atom
   in the preparation of a 4-amino-2-alkylthio-5-pyrimidinecarbaldehyde represented by the following general formula (6):

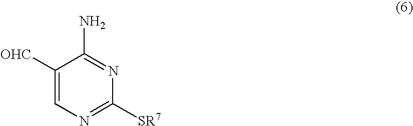

wherein R⁷ represents an alkyl group.

### EFFECT OF THE INVENTION

According to the present invention, there are provided an industrially suitable process for preparing a 4-amino-2-alkylthio-5-pyrimidinecarbaldehyde which can prepare a 4-amino-2-alkylthio-5-pyrimidinecarbaldehyde simply in high yield, an intermediate used in this process, and an industrially suitable process for preparing the intermediate which can prepare the intermediate safely in high yield with ease.

### BEST MODE FOR CARRYING OUT THE INVENTION

In the present invention, an alkyl group means a linear or branched saturated aliphatic hydrocarbon group having 1 to 10 carbon atoms, preferably 1 to 8 carbon atoms, more preferably 1 to 4 carbon atoms. Specific examples of the alkyl groups include a methyl group, an ethyl group, a propyl group, a butyl group, a pentyl group, a hexyl group, a heptyl group, an octyl group, a nonyl group, and a decyl group.

Specific examples of alkali metal atoms include a lithium atom, a sodium atom, a potassium atom, a rubidium atom, and a cesium atom, and preferred examples include a sodium atom and a potassium atom.

Synthesis of Compound (4) from Compound (1) and/or (2)

According to the process of the present invention, at least one nitrile compound selected from the group consisting of compound (1) represented by the following general formula (1):

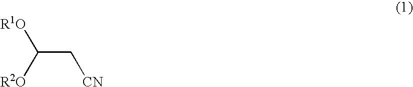

wherein each of R¹ and R² which may be the same or different represents an alkyl group
   and compound (2) represented by the following general formula (2):

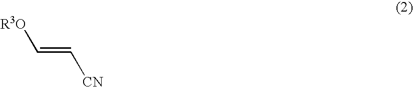

wherein R³ represents an alkyl group,
   and compound (3) represented by the following general formula (3):

HCO₂R⁴ (3)
wherein R⁴ represents an alkyl group,
excluding a methyl group,
   are reacted at −10 to 30° C. in the presence of a base comprising an alkali metal to obtain compound (4) represented by the following general formula (4):

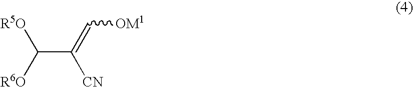

wherein each of R⁵ and R⁶ which may be the same or different represents an alkyl group, and M¹ represents an alkali metal atom.

In compound (1) represented by the general formula (1) above, which is the nitrile compound used in the reaction of the present invention, each of R¹ and R² which may be the same or different represents an alkyl group, and specific examples of the alkyl groups include a methyl group, an ethyl group, a propyl group, a butyl group, a pentyl group, a hexyl group, and a heptyl group, and preferred examples include a methyl group. These groups include their isomers.

As a specific example of compound (1), there can be mentioned 3,3-dimethoxypropanenitrile.

In compound (2) represented by the general formula (2) above, which is the nitrile compound used in the reaction of the present invention, R³ represents an alkyl group, and specific examples of the alkyl groups include a methyl group, an ethyl group, a propyl group, a butyl group, a pentyl group, a hexyl group, and a heptyl group, and preferred examples include a methyl group. These groups include their isomers.

As a specific example of compound (2), there can be mentioned 3-methoxy-2-propenenitrile.

In compound (3) represented by the general formula (3) above used in the reaction of the present invention, R⁴ represents an alkyl group, excluding a methyl group, and specific examples of the alkyl groups include an ethyl group, a propyl group, a butyl group, a pentyl group, a hexyl group, and a heptyl group, and preferred examples include an ethyl group. These groups include their isomers.

As a specific example of compound (3), there can be mentioned ethyl formate having an ethyl group as group R⁴.

The amount of the above formic acid ester used is preferably 0.5 to 5 mol, further preferably 0.8 to 3 mol, relative to 1 mol of the nitrile compound.

Examples of the bases comprising an alkali metal used in the reaction of the present invention include alkali metal hydrides, such as sodium hydride and potassium hydride; lithium amides, such as lithium diisopropylamide and lithium hexamethyldisilazide; alkali metal alkoxides, such as sodium methoxide, sodium t-butoxide, potassium methoxide, and potassium t-butoxide; and alkali metal hydroxides, such as sodium hydroxide and potassium hydroxide, and preferably an alkali metal alkoxide, further preferably sodium methoxide is used. These bases can be used alone or in combination of two or more in admixture as far as they comprise the same alkali metal atom.

The amount of the above base comprising an alkali metal used is preferably 0.5 to 10 mol, further preferably 0.8 to 5 mol, relative to 1 mol of the nitrile compound.

Use of a solvent is preferable in the reaction of the present invention. The solvent to be used is not specifically limited so long as it does not inhibit the reaction, and examples of the solvents include alcohols, such as methanol, ethanol, and isopropyl alcohol; amides, such as N,N-dimethylformamide, N,N-dimethylacetamide, and N-methylpyrrolidone; ureas, such as N,N′-dimethylimidazolidinone; sulfoxides, such as dimethyl sulfoxide; sulfones, such as sulfolane; ethers, such as diethyl ether, diisopropyl ether, tetrahydrofuran, and dioxane; and aromatic hydrocarbons, such as benzene, toluene, and xylene, and preferably an ether or an aromatic hydrocarbon, further preferably tetrahydrofuran or toluene is used. These solvents can be used alone or in combination of two or more in admixture.

The amount of the above-mentioned solvent may be appropriately adjusted depending on the degree of uniformity or condition of stirring of the reaction mixture, and it is preferably 1 to 100 g, further preferably 2 to 50 g, relative to 1 g of the nitrile compound.

The reaction of the present invention may be performed by, for example, a process in which a nitrile compound, a formic acid ester, a base comprising an alkali metal, and a solvent are mixed and reacted with stirring. In this case, the reaction temperature is −10 to 30° C., preferably −5 to 25° C., further preferably −5 to 20° C., and, the reaction pressure is not particularly limited. Compound (1) and compound (2) which are nitrile compounds can be used alone or in combination of two or more in admixture.

As an example of a preferred mode of the reaction of the present invention, there can be mentioned a process in which a nitrile compound and a base comprising an alkali metal are stirred in a solvent and then a formic acid ester is added to the resultant mixture.

In the alkali metal salt of 3,3-dialkoxy-2-hydroxymethylenepropanenitrile represented by the general formula (4) above obtained by the reaction of the present invention, R⁵ and R⁶ are the same as R¹ and R² defined above. M¹ represents an alkali metal atom, and specific examples of the alkali metal atoms include a lithium atom, a sodium atom, and a potassium atom, and preferred examples include a sodium atom.

After the reaction, the alkali metal salt of 3,3-dialkoxy-2-hydroxymethylenepropanenitrile, which is a desired product, is isolated or purified by a general method, such as extraction, filtration, concentration, recrystallization, crystallization, or column chromatography. The reaction mixture containing a product can be directly used in the subsequent reaction without isolating or purifying the resultant alkali metal salt of 3,3-dialkoxy-2-hydroxymethylenepropanenitrile.

Each of compounds (1) to (3) used as starting compounds in the above process is a known compound, and is commercially available or can be easily synthesized by a known method.

### Compound (5)

In compound (5) of the present invention represented by the following general formula (5):

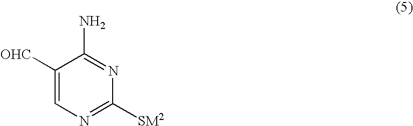

wherein M² represents an alkali metal atom,
   M² represents an alkali metal atom, and specific examples of the alkali metal atoms include a lithium atom, a sodium atom, a potassium atom, a rubidium atom, and a cesium atom, and preferred examples include a sodium atom and a potassium atom.

As specific examples of compounds (5), there can be mentioned the following compounds:

a sodium salt of 4-amino-2-mercapto-5-pyrimidinecarbaldehyde; and
a potassium salt of 4-amino-2-mercapto-5-pyrimidinecarbaldehyde.

This compound is a novel compound, and the alkali metal salt has excellent filtering properties and is easy to isolate, and hence it is very easy to handle in the reaction step, and, as mentioned below, a 4-amino-2-alkylthio-5-pyrimidinecarbaldehyde, which is a compound advantageously used as a starting material or a synthetic intermediate for pharmaceuticals or pesticides, can be easily derived from the compound.

Synthesis of Compound (5) from Compound (4)

Compound (5) can be obtained by the process of the present invention by reacting compound (4), which is obtained by the above-mentioned process, and which is represented by the following general formula (4):

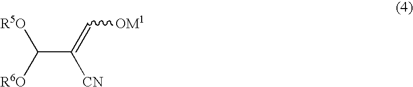

wherein each of R⁵ and R⁶ which may be the same or different represents an alkyl group, and M¹ represents an alkali metal atom,
   with thiourea.

In compound (4) used in the reaction of the present invention, each of R⁵ and R⁶ which may be the same or different represents an alkyl group, and specific examples of the alkyl groups include a methyl group, an ethyl group, a propyl group, a butyl group, a pentyl group, a hexyl group, and a heptyl group, and preferred examples include a methyl group and an ethyl group. These groups include their isomers.

M¹ represents an alkali metal atom and may be the same or different from M², and specific examples of the alkali metal atoms include a lithium atom, a sodium atom, a potassium atom, a rubidium atom, and a cesium atom, and preferred examples include a sodium atom and a potassium atom.

As specific examples of compounds (4), there can be mentioned the following compounds:

a sodium salt of 3,3-diethoxy-2-hydroxymethylenepropanenitrile;
a sodium salt of 3-ethoxy-3-methoxy-2-hydroxymethylenepropanenitrile; and
a sodium salt of 3,3-dimethoxy-2-hydroxymethylenepropanenitrile.

The amount of the thiourea used in the reaction of the present invention is preferably 0.5 to 10 mol, further preferably 0.8 to 5.0 mol, relative to 1 mol of compound (4).

It is desired that the reaction of the present invention is conducted in a solvent in the presence of a base.

Examples of the bases used in the reaction of the present invention include alkali metal hydrides, such as sodium hydride and potassium hydride; lithium amides, such as lithium diisopropylamide and lithium hexamethyldisilazide; alkali metal alkoxides, such as sodium methoxide, sodium t-butoxide, potassium methoxide, and potassium t-butoxide; alkali metal hydroxides, such as sodium hydroxide and potassium hydroxide; alkali metal carbonates, such as sodium carbonate and potassium carbonate; and alkali metal hydrogencarbonates, such as sodium hydrogencarbonate and potassium hydrogencarbonate, and preferably an alkali metal alkoxide, further preferably sodium methoxide or potassium methoxide is used. These bases can be used alone or in combination of two or more in admixture as far as they comprise the same alkali metal atom.

The amount of the above base used is preferably 0.1 to 10 mol, further preferably 0.1 to 5 mol, relative to 1 mol of compound (4).

When the reaction mixture obtained in the previous step for preparing compound (4) from compound (1) and/or compound (2) is directly used in the reaction for preparing compound (5), the base comprising an alkali metal used in the previous step is present in the reaction mixture, and therefore it may not be necessary to add a base in the following step.

The solvent used in the reaction of the present invention is not particularly limited so long as it does not inhibit the reaction, and examples of the solvents include alcohols, such as methanol, ethanol, isopropyl alcohol, t-butyl alcohol, methoxyethanol, ethoxyethanol, and butoxyethanol; nitrites, such as acetonitrile, propionitrile, and benzonitrile; amides, such as N,N-dimethylformamide, N,N-dimethylacetamide, and N-methylpyrrolidone; ureas, such as N,N′-dimethylimidazolidinone; sulfoxides, such as dimethyl sulfoxide; sulfones, such as sulfolane; ethers, such as diethyl ether, diisopropyl ether, tetrahydrofuran, and dioxane; and aromatic hydrocarbons, such as benzene, toluene, and xylene, and preferably an alcohol, an ether, or an aromatic hydrocarbon, further preferably methanol, ethanol, isopropyl alcohol, butoxyethanol, tetrahydrofuran, or toluene is used. These solvents can be used alone or in combination of two or more in admixture.

The amount of the above solvent used is appropriately selected depending on the uniformity or stirring properties of the reaction mixture, but it is preferably 0.1 to 100 g, further preferably 0.5 to 50 g, relative to 1 g of compound (4).

The reaction of the present invention is performed by, for example, a process in which compound (4), thiourea, and optionally a base and a solvent are mixed together and reacted while stirring. In this case, the reaction temperature is preferably 0 to 200° C., further preferably 0 to 150° C., and the reaction pressure is not particularly limited.

Compound (5) is obtained by the reaction of the present invention, and has excellent filtering properties and is easy to isolate and hence, after the reaction, this compound is easily isolated or purified by a general method, such as extraction, filtration, concentration, recrystallization, crystallization, or column chromatography.

Synthesis of Compound (6) from Compound (5)

Compound (6) represented by the following general formula (6):

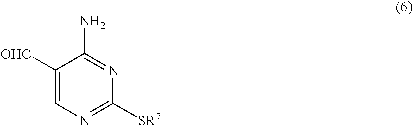

wherein R⁷ represents an alkyl group
   can be obtained by the process of the present invention by reacting compound (5), which is obtained by the above-mentioned process, and which is represented by the following general formula (5):

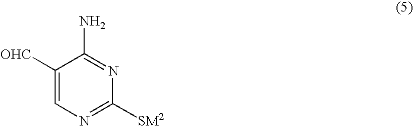

wherein M² represents an alkali metal atom,
   with an alkylating agent.

In compound (5) used in the process of the present invention, M² represents an alkali metal atom, and specific examples of the alkali metal atoms include a lithium atom, a sodium atom, a potassium atom, a rubidium atom, and a cesium atom, and preferred examples include a sodium atom and a potassium atom.

The alkylating agent used in the reaction of the present invention is not particularly limited so long as it can derive compound (6) from compound (5) by alkylation, namely, by introducing a desired alkyl group R⁷, and examples of the alkylating agents include alkyl halides, such as methyl iodide and ethyl bromide; alkyl organosulfonates, such as methyl methanesulfonate, methyl trifluoromethanesulfonate, and methyl p-toluenesulfonate; and dialkyl sulfates, such as dimethyl sulfate and diethyl sulfate, and preferably an alkyl halide or a dialkyl sulfate, further preferably methyl iodide or dimethyl sulfate is used. These alkylating agents can be used in combination of two or more in admixture as far as they introduce the same alkyl group in the alkylation.

The amount of the alkylating agent used in the reaction of the present invention is preferably 0.5 to 10 equivalent amount, further preferably 0.8 to 5 equivalent amount, relative to 1 mol of compound (5).

It is desired that the reaction of the present invention is conducted in the presence of a solvent. The solvent used is not particularly limited so long as it does not inhibit the reaction, and examples of the solvents include water; alcohols, such as methanol, ethanol, isopropyl alcohol, t-butyl alcohol, methoxyethanol, ethoxyethanol, and butoxyethanol; nitriles, such as acetonitrile, propionitrile, and benzonitrile; ketones, such as acetone, methyl ethyl ketone, and methyl isobutyl ketone; amides, such as N,N-dimethylformamide, N,N-dimethylacetamide, and N-methylpyrrolidone; ureas, such as N,N′-dimethylimidazolidinone; sulfoxides, such as dimethyl sulfoxide; and sulfones, such as sulfolane, and preferably water or an alcohol, further preferably water or methanol is used. These solvents can be used alone or in combination of two or more in admixture.

The amount of the above solvent used is appropriately selected depending on the uniformity or stirring properties of the reaction mixture, but it is preferably 0.1 to 100 g, further preferably 0.5 to 50 g, relative to 1 g of compound (5).

The reaction of the present invention is performed by, for example, a process in which compound (5), an alkylating agent, and a solvent are mixed together and reacted while stirring. In this case, the reaction temperature is preferably −30 to 200° C., further preferably −20 to 150° C., and, the reaction pressure is not particularly limited.

Compound (6) is obtained by the reaction of the present invention, and, after the reaction, this compound is isolated or purified by a general method, such as neutralization, extraction, filtration, concentration, distillation, recrystallization, crystallization, or column chromatography.

### EXAMPLES

Hereinbelow, the present invention will be described in more detail with reference to the following Examples, which should not be construed as limiting the scope of the present invention. An alkali metal salt of 3,3-dialkoxy-2-hydroxymethylenepropanenitrile {compound (4)}, which is a desired product, decomposes to 2-cyanomalonaldehyde during the analysis by high performance liquid chromatography, and therefore it was quantitatively determined as 2-cyanomalonaldehyde to determine a reaction yield.

### Example 1

### Synthesis of compound (4) (Synthesis of sodium salt of 3,3-diethoxy-2-hydroxymethylenepropanenitrile, sodium salt of 3-ethoxy-3-methoxy-2-hydroxymethylenepropanenitrile, and sodium salt of 3,3-dimethoxy-2-hydroxymethylenepropanenitrile)

Into a flask made of glass having an inner volume of 100 ml and equipped with a stirring device, a thermometer, and a dropping funnel were placed 11.51 g (100 mmol) of 3,3-dimethoxypropanenitrile, 10.8 g (200 mmol) of sodium methoxide, and 35 ml of toluene. While maintaining the liquid temperature at 15 to 20° C., a solution comprising 9.30 g (122 mmol) of 97% by mass ethyl formate and 12 ml of toluene was added slowly and the mixture was reacted under stirring at the same temperature for 8 hours. After completion of the reaction, the resultant reaction solution was analyzed by high performance liquid chromatography (absolute quantitative determination method). As a result, it was found that a sodium salt of 3,3-diethoxy-2-hydroxymethylenepropanenitrile, a sodium salt of 3-ethoxy-3-methoxy-2-hydroxymethylenepropanenitrile, and a sodium salt of 3,3-dimethoxy-2-hydroxymethylenepropanenitrile were formed in a total amount of 93.3 mmol (reaction yield based on 3,3-dimethoxypropanenitrile: 93.3%). The amount of carbon monoxide generated in this reaction was as small as 4.7 mmol (generation rate based on ethyl formate: 3.9%).

### Example 2

### Synthesis of compound (4) (Synthesis of sodium salt of 3,3-diethoxy-2-hydroxymethylenepropanenitrile, sodium salt of 3-ethoxy-3-methoxy-2-hydroxymethylenepropanenitrile, and sodium salt of 3,3-dimethoxy-2-hydroxymethylenepropanenitrile)

Into a flask made of glass having an inner volume of 100 ml and equipped with a stirring device, a thermometer, and a dropping funnel were placed 11.51 g (100 mmol) of 3,3-dimethoxypropanenitrile, 10.8 g (200 mmol) of sodium methoxide, and 35 ml of toluene. While maintaining the liquid temperature at 10 to 15° C., a solution comprising 9.30 g (122 mmol) of 97% by mass ethyl formate and 12 ml of toluene was added slowly and the mixture was reacted under stirring at the same temperature for 8 hours. After completion of the reaction, the resultant reaction solution was analyzed by high performance liquid chromatography (absolute quantitative determination method). As a result, it was found that a sodium salt of 3,3-diethoxy-2-hydroxymethylenepropanenitrile, a sodium salt of 3-ethoxy-3-methoxy-2-hydroxymethylenepropanenitrile, and a sodium salt of 3,3-dimethoxy-2-hydroxymethylenepropanenitrile were formed in a total amount of 89.2 mmol (reaction yield based on 3,3-dimethoxypropanenitrile: 89.2%). The amount of carbon monoxide generated in this reaction was as small as 3.0 mmol (generation rate based on ethyl formate: 2.5%).

### Comparative Example 1

### Synthesis of sodium salt of 3,3-diethoxy-2-hydroxymethylenepropanenitrile, sodium salt of 3-ethoxy-3-methoxy-2-hydroxymethylenepropanenitrile, and sodium salt 3,3-dimethoxy-2-hydroxymethylenepropanenitrile

Into a flask made of glass having an inner volume of 25 ml and equipped with a stirring device, a thermometer, and a dropping funnel were placed 1.15 g (10 mmol) of 3,3-dimethoxypropanenitrile, 1.08 g (20 mmol) of sodium methoxide, and 3.5 ml of toluene. While maintaining the liquid temperature at 35 to 40° C., a solution comprising 0.93 g (12.2 mmol) of 97% by mass ethyl formate and 1.2 ml of toluene was added slowly and the mixture was reacted under stirring at the same temperature for 6 hours. After completion of the reaction, the resultant reaction solution was analyzed by high performance liquid chromatography (absolute quantitative determination method). As a result, it was found that a sodium salt of 3,3-diethoxy-2-hydroxymethylenepropanenitrile, a sodium salt of 3-ethoxy-3-methoxy-2-hydroxymethylenepropanenitrile, and a sodium salt of 3,3-dimethoxy-2-hydroxymethylenepropanenitrile were formed in a total amount of 9.32 mmol (reaction yield based on 3,3-dimethoxypropanenitrile: 93.2%). The amount of carbon monoxide generated in this reaction was 1.2 mmol (generation rate based on ethyl formate: 9.8%).

### Comparative Example 2

### Synthesis of sodium salt of 3,3-dimethoxy-2-hydroxymethylenepropanenitrile

Into a flask made of glass having an inner volume of 25 ml and equipped with a stirring device, a thermometer, and a dropping funnel were placed 1.15 g (10 mmol) of 3,3-dimethoxypropanenitrile, 1.08 g (20 mmol) of sodium methoxide, and 3.5 ml of toluene. While maintaining the liquid temperature at 35 to 40° C., a solution comprising 0.76 g (12.2 mmol) of 97% by mass methyl formate and 1.2 ml of toluene was added slowly and the mixture was reacted under stirring at the same temperature for 6 hours. After completion of the reaction, the resultant reaction solution was analyzed by high performance liquid chromatography (absolute quantitative determination method). As a result, it was found that a sodium salt of 3,3-dimethoxy-2-hydroxymethylenepropanenitrile was formed in an amount of 5.50 mmol (reaction yield based on 3,3-dimethoxypropanenitrile: 55.0%). The amount of carbon monoxide generated in this reaction was 2.8 mmol (generation rate based on methyl formate: 23.0%).

### Example 3

### Synthesis of compound (4) (Synthesis of sodium salt of 3,3-diethoxy-2-hydroxymethylenepropanenitrile, sodium salt of 3-ethoxy-3-methoxy-2-hydroxymethylenepropanenitrile, and sodium salt of 3,3-dimethoxy-2-hydroxymethylenepropanenitrile)

Into a flask made of glass having an inner volume of 100 ml and equipped with a stirring device, a thermometer, and a dropping funnel were placed 8.3 g (100 mmol) of 3-methoxy-2-propenenitrile, 10.8 g (200 mmol) of sodium methoxide, and 30 ml of tetrahydrofuran. While maintaining the liquid temperature at 0 to 10° C., a solution comprising 9.16 g (120 mmol) of 97% by mass ethyl formate and 10 ml of tetrahydrofuran was added slowly and the mixture was reacted under stirring at the same temperature for 6 hours. After completion of the reaction, the resultant reaction solution was analyzed by high performance liquid chromatography (absolute quantitative determination method). As a result, it was found that a sodium salt of 3,3-diethoxy-2-hydroxymethylenepropanenitrile, a sodium salt of 3-ethoxy-3-methoxy-2-hydroxymethylenepropanenitrile, and a sodium salt of 3,3-dimethoxy-2-hydroxymethylenepropanenitrile were formed in a total amount of 94.9 mmol (reaction yield based on 3-methoxy-2-propenenitrile: 94.9%). The amount of carbon monoxide generated in this reaction was as small as 4.9 mmol (generation rate based on ethyl formate: 4.1%).

### Comparative Example 3

### Synthesis of sodium salt of 3,3-diethoxy-2-hydroxymethylenepropanenitrile, sodium salt of 3-ethoxy-3-methoxy-2-hydroxymethylenepropanenitrile, and sodium salt of 3,3-dimethoxy-2-hydroxymethylenepropanenitrile

Into a flask made of glass having an inner volume of 25 ml and equipped with a stirring device, a thermometer, and a dropping funnel were placed 0.83 g (10 mmol) of 3-methoxy-2-propenenitrile, 1.08 g (20 mmol) of sodium methoxide, and 3.5 ml of toluene. While maintaining the liquid temperature at 35 to 40° C., a solution comprising 0.93 g (12.2 mmol) of 97% by mass ethyl formate and 1.2 ml of toluene was added slowly and the mixture was reacted under stirring at the same temperature for 6 hours. After completion of the reaction, the resultant reaction solution was analyzed by high performance liquid chromatography (absolute quantitative determination method). As a result, it was found that a sodium salt of 3,3-diethoxy-2-hydroxymethylenepropanenitrile, a sodium salt of 3-ethoxy-3-methoxy-2-hydroxymethylenepropanenitrile, and a sodium salt of 3,3-dimethoxy-2-hydroxymethylenepropanenitrile were formed in a total amount of 8.90 mmol (reaction yield based on 3-methoxypropenenitrile: 89.0%). The amount of carbon monoxide generated in this reaction was 1.4 mmol (generation rate based on ethyl formate: 11.46).

### Example 4

### Synthesis of compound (4) (Synthesis of sodium salt of 3,3-diethoxy-2-hydroxymethylenepropanenitrile, sodium salt of 3-ethoxy-3-methoxy-2-hydroxymethylenepropanenitrile, and sodium salt of 3,3-dimethoxy-2-hydroxymethylenepropanenitrile)

Into a flask made of glass having an inner volume of 100 ml and equipped with a stirring device, a thermometer, and a dropping funnel were placed 11.51 g (100 mmol) of 3,3-dimethoxypropanenitrile, 10.8 g (200 mmol) of sodium methoxide, and 20 ml of tetrahydrofuran. While maintaining the liquid temperature at 0 to 5° C., a solution comprising 9.16 g (120 mmol) of 97% by mass ethyl formate and 10 ml of tetrahydrofuran was added slowly and the mixture was reacted under stirring at the same temperature for 6 hours. After completion of the reaction, the resultant reaction solution was analyzed by high performance liquid chromatography (absolute quantitative determination method). As a result, it was found that a sodium salt of 3,3-diethoxy-2-hydroxymethylenepropanenitrile, a sodium salt of 3-ethoxy-3-methoxy-2-hydroxymethylenepropanenitrile, and a sodium salt of 3,3-dimethoxy-2-hydroxymethylenepropanenitrile were formed in a total amount of 97.8 mmol (reaction yield based on 3,3-dimethoxypropanenitrile: 97.8%). The amount of carbon monoxide generated in this reaction was as small as 3.0 mmol (generation rate based on ethyl formate: 2.5%).

### Example 5

### Synthesis of compound (4) (Synthesis of sodium salt of 3,3-diethoxy-2-hydroxymethylenepropanenitrile, sodium salt of 3-ethoxy-3-methoxy-2-hydroxymethylenepropanenitrile, and sodium salt of 3,3-dimethoxy-2-hydroxymethylenepropanenitrile)

Into a flask made of glass having an inner volume of 100 ml and equipped with a stirring device, a thermometer, and a dropping funnel were placed 11.51 g (100 mmol) of 3,3-dimethoxypropanenitrile, 10.8 g (200 mmol) of sodium methoxide, and 30 ml of tetrahydrofuran. While maintaining the liquid temperature at 10 to 15° C., a solution comprising 9.16 g (120 mmol) of 97% by mass ethyl formate and 10 ml of tetrahydrofuran was added slowly and the mixture was reacted under stirring at the same temperature for 6 hours. After completion of the reaction, the resultant reaction solution was analyzed by high performance liquid chromatography (absolute quantitative determination method). As a result, it was found that a sodium salt of 3,3-diethoxy-2-hydroxymethylenepropanenitrile, a sodium salt of 3-ethoxy-3-methoxy-2-hydroxymethylenepropanenitrile, and a sodium salt of 3,3-dimethoxy-2-hydroxymethylenepropanenitrile were formed in a total amount of 97.0 mmol (reaction yield based on 3,3-dimethoxypropanenitrile: 97.0%). The amount of carbon monoxide generated in this reaction was as small as 5.9 mmol (generation rate based on ethyl formate: 4.9%).

### Example 6

### Synthesis of compound (4) (Synthesis of sodium salt of 3,3-diethoxy-2-hydroxymethylenepropanenitrile, sodium salt of 3-ethoxy-3-methoxy-2-hydroxymethylenepropanenitrile, and sodium salt of 3,3-dimethoxy-2-hydroxymethylenepropanenitrile)

A reaction was conducted in substantially the same manner as in Example 1 except that, instead of 3,3-dimethoxypropanenitrile, a 1:1 (molar ratio) mixture of 3,3-dimethoxypropanenitrile and 3-methoxy-2-propenenitrile was used. As a result, a sodium salt of 3,3-diethoxy-2-hydroxymethylenepropanenitrile, a sodium salt of 3-ethoxy-3-methoxy-2-hydroxymethylenepropanenitrile, and a sodium salt of 3,3-dimethoxy-2-hydroxymethylenepropanenitrile were obtained in high yield, and the amount of carbon monoxide generated in this reaction was small.

### Example 7

### Synthesis of compound (5) (M²=sodium atom) (Synthesis of sodium salt of 4-amino-2-mercapto-5-pyrimidinecarbaldehyde)

Into a flask made of glass having an inner volume of 200 ml and equipped with a stirring device, a thermometer, a dropping funnel, and a reflux condenser were placed 11.51 g (100 mmol) of 3,3-dimethoxypropanenitrile, 10.80 g (200 mmol) of sodium methoxide, and 20 ml of tetrahydrofuran. While maintaining the liquid temperature at 5 to 10° C., a solution comprising 9.16 g (120 mmol) of 97% by mass ethyl formate and 10 ml of tetrahydrofuran was added slowly and the mixture was reacted under stirring at the same temperature for 4.5 hours, as a result, a solution containing a sodium salt of 3,3-dimethoxy-2-hydroxymethylenepropanenitrile as a main component was obtained.

To the above solution containing a sodium salt of 3,3-dimethoxy-2-hydroxymethylenepropanenitrile as a main component were added 7.99 g (105 mmol) of thiourea, 25 ml of 2-butoxyethanol, and 30 ml of isopropyl alcohol and the mixture was reacted under stirring at 50° C. for 3 hours.

After completion of the reaction, the resultant reaction solution was concentrated under a reduced pressure, and then 11.2 ml of methanol and 37.5 ml of water were added to the resultant concentrate and stirred at 20 to 25° C. for 1 hour. The resultant solids were collected by filtration, and then dried under a reduced pressure to obtain 13.28 g of 94.5% by mass (value quantitatively determined by high performance liquid chromatography) sodium salt of 4-amino-2-mercapto-5-pyrimidinecarbaldehyde, as yellow powder (isolation yield based on 3,3-dimethoxypropanenitrile: 70.8%).

The sodium salt of 4-amino-2-mercapto-5-pyrimidinecarbaldehyde is a novel compound having the following physical properties.

Melting point: 297 to 300° C.

¹H-NMR {DMSO-d₆, δ (ppm)}: 6.75 to 7.70 (2H, brs), 7.99 (1H, s), 9.38 (1H, s)

### Example 8

### Synthesis of compound (5) (M²=potassium atom) (Synthesis of potassium salt of 4-amino-2-mercapto-5-pyrimidinecarbaldehyde)

Into a flask made of glass having an inner volume of 200 ml and equipped with a stirring device, a thermometer, a dropping funnel, and a reflux condenser were placed 11.51 g (100 mmol) of 3,3-dimethoxypropanenitrile, 14.77 g (200 mmol) of 95% by mass potassium methoxide, and 50 ml of tetrahydrofuran. While maintaining the liquid temperature at 5 to 10° C., a solution comprising 9.16 g (120 mmol) of 97% by mass ethyl formate and 10 ml of tetrahydrofuran was added slowly and the mixture was reacted under stirring at the same temperature for 4.5 hours, as a result, a solution containing a potassium salt of 3,3-dimethoxy-2-hydroxymethylenepropanenitrile as a main component was obtained.

To the above solution containing a potassium salt of 3,3-dimethoxy-2-hydroxymethylenepropanenitrile as a main component were added 7.99 g (105 mmol) of thiourea, 25 ml of 2-butoxyethanol, and 30 ml of isopropyl alcohol and the mixture was reacted under stirring at 50° C. for 3 hours.

After completion of the reaction, the resultant reaction solution was concentrated under a reduced pressure, and then 11.2 ml of methanol and 37.5 ml of water were added to the resultant concentrate and stirred at 20 to 25° C. for 1 hour. The resultant solids were collected by filtration, and then dried under a reduced pressure to obtain 7.63 g of 99.0% by mass (value quantitatively determined by high performance liquid chromatography) potassium salt of 4-amino-2-mercapto-5-pyrimidinecarbaldehyde, as pale yellow powder (isolation yield based on 3,3-dimethoxypropanenitrile: 39.0%).

The potassium salt of 4-amino-2-mercapto-5-pyrimidinecarbaldehyde is a novel compound having the following physical properties.

Melting point: 303 to 305° C.

¹H-NMR {DMSO-d₆, δ (ppm)}: 6.80 to 7.70 (2H, brs), 7.99 (1H, s), 9.36 (1H, s)

### Example 9

### Synthesis of compound (5) (M²=sodium atom) (Synthesis of sodium salt of 4-amino-2-mercapto-5-pyrimidinecarbaldehyde)

A solution containing a sodium salt of 3,3-dimethoxy-2-hydroxymethylenepropanenitrile as a main component was obtained in the same manner as in Example 7.

To the resultant solution containing a sodium salt of 3,3-dimethoxy-2-hydroxymethylenepropanenitrile as a main component were added 7.99 g (105 mmol) of thiourea, 25 ml of 2-butoxyethanol, and 30 ml of methanol and the mixture was reacted under stirring at 50° C. for 3 hours.

After completion of the reaction, the resultant reaction solution was concentrated under a reduced pressure, and then 37.5 ml of water was added to the resultant concentrate and stirred at 20 to 25° C. for 1 hour. The resultant solids were collected by filtration, and then dried under a reduced pressure to obtain 9.89 g of 98.4% by mass (value quantitatively determined by high performance liquid chromatography) sodium salt of 4-amino-2-mercapto-5-pyrimidinecarbaldehyde, as yellow powder (isolation yield based on 3,3-dimethoxypropanenitrile: 54.9%).

### Example 10

### Synthesis of compound (5) (M²=sodium atom) (Synthesis of sodium salt of 4-amino-2-mercapto-5-pyrimidinecarbaldehyde)

A solution containing a sodium salt of 3,3-dimethoxy-2-hydroxymethylenepropanenitrile as a main component was obtained in the same manner as in Example 7.

To the resultant solution containing a sodium salt of 3,3-dimethoxy-2-hydroxymethylenepropanenitrile as a main component were added 7.99 g (105 mmol) of thiourea, 25 ml of 2-butoxyethanol, and 30 ml of ethanol and the mixture was reacted under stirring at 50° C. for 3 hours.

After completion of the reaction, the resultant reaction solution was concentrated under a reduced pressure, and then 11.2 ml of methanol and 37.5 ml of water were added to the resultant concentrate and stirred at 20 to 25° C. for 1 hour. The resultant solids were collected by filtration, and then dried under a reduced pressure to obtain 13.05 g of 96.5% by mass (value quantitatively determined by high performance liquid chromatography) sodium salt of 4-amino-2-mercapto-5-pyrimidinecarbaldehyde, as yellow powder (isolation yield based on 3,3-dimethoxypropanenitrile: 71.0%).

### Example 11

### Synthesis of compound (5) (M²=sodium atom) (Synthesis of sodium salt of 4-amino-2-mercapto-5-pyrimidinecarbaldehyde)

A solution containing a sodium salt of 3,3-dimethoxy-2-hydroxymethylenepropanenitrile as a main component was obtained in the same manner as in Example 7.

To the resultant solution containing a sodium salt of 3,3-dimethoxy-2-hydroxymethylenepropanenitrile as a main component were added 7.99 g (105 mmol) of thiourea and 55 ml of 2-butoxyethanol and the mixture was reacted under stirring at 50° C. for 3 hours.

After completion of the reaction, the resultant reaction solution was concentrated under a reduced pressure, and then 11.2 ml of methanol and 37.5 ml of water were added to the resultant concentrate and stirred at 20 to 25° C. for 1 hour. The resultant solids were collected by filtration, and then dried under a reduced pressure to obtain 12.40 g of 96.0% by mass (value quantitatively determined by high performance liquid chromatography) sodium salt of 4-amino-2-mercapto-5-pyrimidinecarbaldehyde, as yellow powder (isolation yield based on 3,3-dimethoxypropanenitrile: 67.2%).

### Example 12

### Synthesis of compound (5) (M²=sodium atom) (Synthesis of sodium salt of 4-amino-2-mercapto-5-pyrimidinecarbaldehyde)

A solution containing a sodium salt of 3,3-dimethoxy-2-hydroxymethylenepropanenitrile as a main component was obtained in the same manner as in Example 7.

To the resultant solution containing a sodium salt of 3,3-dimethoxy-2-hydroxymethylenepropanenitrile as a main component were added 7.99 g (105 mmol) of thiourea and 55 ml of isopropyl alcohol and the mixture was reacted under stirring at 50° C. for 3 hours.

After completion of the reaction, the resultant reaction solution was concentrated under a reduced pressure, and then 11.2 ml of methanol and 37.5 ml of water were added to the resultant concentrate and stirred at 20 to 25° C. for 1 hour. The resultant solids were collected by filtration, and then dried under a reduced pressure to obtain 14.88 g of 78.4% by mass (value quantitatively determined by high performance liquid chromatography) sodium salt of 4-amino-2-mercapto-5-pyrimidinecarbaldehyde, as yellow powder (isolation yield based on 3,3-dimethoxypropanenitrile: 65.8%).

### Example 13

### Synthesis of compound (6) (R⁷=methyl group) (Synthesis of 4-amino-2-methylthio-5-pyrimidinecarbaldehyde)

Into a flask made of glass having an inner volume of 2,000 ml and equipped with a stirring device, a thermometer, and a dropping funnel were placed 200.0 g (564 mmol) of 50.0% by mass sodium salt of 4-amino-2-mercapto-5-pyrimidinecarbaldehyde synthesized in the same manner as in Example 7, 325 ml of methanol, and 225 ml of water, and then, while maintaining the liquid temperature at 15 to 25° C., 92.7 g (620 mmol) of 95% by mass methyl iodide was added slowly and the mixture was reacted under stirring at the same temperature for 2 hours. After completion of the reaction, the crystals deposited were collected by filtration, and dried under a reduced pressure to obtain 99.6 g of 94.2% by mass (value quantitatively determined by high performance liquid chromatography) 4-amino-2-methylthio-5-pyrimidinecarbaldehyde, as yellow crystals (isolation yield based on sodium salt of 4-amino-2-mercapto-5-pyrimidinecarbaldehyde: 98.3%).

The physical properties of 4-amino-2-methylthio-5-pyrimidinecarbaldehyde were as follows.

CI-MS (m/e): 170(M+1)

¹H-NMR {DMSO-d₆, δ (ppm)}: 2.50 (3H, s), 8.03 (1H, brs), 8.28 (1H, brs), 8.57 (1H, s), 9.77 (1H, s)

### Example 14

### Synthesis of compound (6) (R⁷=methyl group) (Synthesis of 4-amino-2-methylthio-5-pyrimidinecarbaldehyde)

Into a flask made of glass having an inner volume of 200 ml and equipped with a stirring device, a thermometer, and a dropping funnel were placed 20.0 g (56.4 mmol) of 50.0% by mass sodium salt of 4-amino-2-mercapto-5-pyrimidinecarbaldehyde synthesized in the same manner as in Example 7 and 55 ml of water, and then, while maintaining the liquid temperature at 15 to 25° C., 10.1 g (67.6 mmol) of 95% by mass methyl iodide was added slowly and the mixture was reacted under stirring at the same temperature for 2 hours. After completion of the reaction, the crystals deposited were collected by filtration, and dried under a reduced pressure to obtain 9.84 g of 94.7% by mass (value quantitatively determined by high performance liquid chromatography) 4-amino-2-methylthio-5-pyrimidinecarbaldehyde, as yellow crystals (isolation yield based on sodium salt of 4-amino-2-mercapto-5-pyrimidinecarbaldehyde: 97.6%).

### Example 15

### Synthesis of compound (6) (R⁷=methyl group) (Synthesis of 4-amino-2-methylthio-5-pyrimidinecarbaldehyde)

Into a flask made of glass having an inner volume of 200 ml and equipped with a stirring device, a thermometer, and a dropping funnel were placed 18.0 g (56.6 mmol) of 55.7% by mass sodium salt of 4-amino-2-mercapto-5-pyrimidinecarbaldehyde synthesized in the same manner as in Example 7 and 59.5 ml of water, and then, while maintaining the liquid temperature at 15 to 30° C., 8.2 g (61.8 mmol) of 95% by mass dimethyl sulfate was added slowly and the mixture was reacted under stirring at the same temperature for 1 hour. After completion of the reaction, the crystals deposited were collected by filtration, and dried under a reduced pressure to obtain 8.34 g of 92.1% by mass (value quantitatively determined by high performance liquid chromatography) 4-amino-2-methylthio-5-pyrimidinecarbaldehyde, as yellow crystals (isolation yield based on sodium salt of 4-amino-2-mercapto-5-pyrimidinecarbaldehyde: 80.2%).

### Test Example 1

### Filtering properties of compound (5) (M²=sodium atom) (sodium salt of 4-amino-2-mercapto-5-pyrimidinecarbaldehyde)

300 ml of the reaction solution containing 39.3 g of a sodium salt of 4-amino-2-mercapto-5-pyrimidinecarbaldehyde synthesized in the same manner as in Example 7 was subjected to filtration under a reduced pressure of 4.8×10⁴ Pa using a glass filter having a diameter of 2.8×10⁻³ m² equipped with filter paper (5C; manufactured by Toyo Roshi Kaisha, Ltd.). The filtration was completed in about 59 seconds.

### Test Example 2

### Filtering properties of 4-amino-2-mercapto-5-pyrimidinecarbaldehyde

300 ml of the reaction solution containing 39.3 g of a sodium salt of 4-amino-2-mercapto-5-pyrimidinecarbaldehyde synthesized in the same manner as in Example 7 was neutralized by adding water and sulfuric acid to obtain 300 ml of the reaction solution containing 27.2 g of 4-amino-2-mercapto-5-pyrimidinecarbaldehyde, and the reaction solution was subjected to filtration under a reduced pressure of 4.8×10⁴ Pa using a glass filter having a diameter of 2.8×10⁻³ m² equipped with filter paper (5C; manufactured by Toyo Roshi Kaisha, Ltd.). The filtration required a period of time so long as about 414 seconds.

### INDUSTRIAL APPLICABILITY

According to the present invention, there are provided an industrially suitable process for preparing a 4-amino-2-alkylthio-5-pyrimidinecarbaldehyde which can prepare a 4-amino-2-alkylthio-5-pyrimidinecarbaldehyde simply in high yield, an intermediate used in this process, and an industrially suitable process for preparing the intermediate which can prepare the intermediate safely in high yield with ease.

1. A process for preparing an alkali metal salt of 3,3-dialkoxy-2-hydroxymethylenepropanenitrile represented by the following general formula (4): 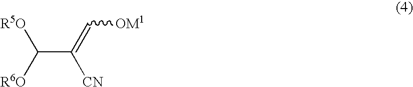
wherein each of R⁵ and R⁶ which may be the same or different represents an alkyl group, and M¹ represents an alkali metal atom,
the process comprising reacting at least one nitrile compound selected from the group consisting of a 3,3-dialkoxypropanenitrile represented by the following general formula (1): 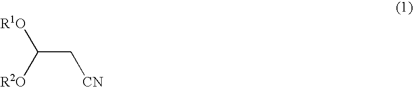
wherein each of R¹ and R² which may be the same or different represents an alkyl group
and a 3-alkoxy-2-propenenitrile represented by the following general formula (2): 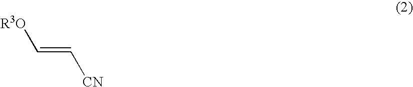
wherein R³ represents an alkyl group,
with a formic acid ester represented by the following general formula (3):
HCO₂R⁴ (3)
wherein R⁴ represents an alkyl group, excluding a methyl group,
at −10 to 30° C. in the presence of a base comprising an alkali metal.

2. The process for preparing an alkali metal salt of 3,3-dialkoxy-2-hydroxymethylenepropanenitrile according to claim 1, wherein the reaction is conducted at −5 to 20° C.

3. The process for preparing an alkali metal salt of 3,3-dialkoxy-2-hydroxymethylenepropanenitrile according to claim 1, wherein the base comprising an alkali metal is a base comprising a sodium atom or a potassium atom.

4. The process for preparing an alkali metal salt of 3,3-dialkoxy-2-hydroxymethylenepropanenitrile according to claim 1, wherein R⁴ is an ethyl group.

5. An alkali metal salt of 4-amino-2-mercapto-5-pyrimidinecarbaldehyde represented by the following general formula (5): 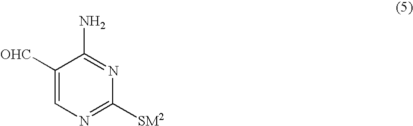
wherein M² represents an alkali metal atom.

6. A sodium salt or potassium salt of 4-amino-2-mercapto-5-pyrimidinecarbaldehyde.

7. A process for preparing an alkali metal salt of 4-amino-2-mercapto-5-pyrimidinecarbaldehyde represented by the following general formula (5): 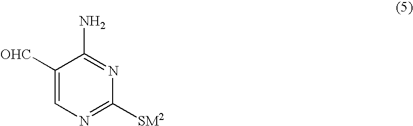
wherein M² represents an alkali metal atom,
the process comprising reacting an alkali metal salt of 3,3-dialkoxy-2-hydroxymethylenepropanenitrile represented by the following general formula (4): 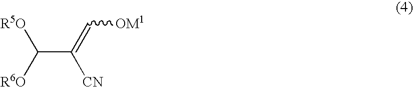
wherein each of R⁵ and R⁶ which may be the same or different represents an alkyl group, and M¹ represents an alkali metal atom
with thiourea.

8. The process for preparing an alkali metal salt of 4-amino-2-mercapto-5-pyrimidinecarbaldehyde according to claim 7, wherein the reaction is conducted in a solvent in the presence of a base.

9. The process for preparing an alkali metal salt of 4-amino-2-mercapto-5-pyrimidinecarbaldehyde according to claim 7, further comprising a step for obtaining an alkali metal salt of 3,3-dialkoxy-2-hydroxymethylenepropanenitrile by the process.

10. A process for preparing a 4-amino-2-alkylthio-5-pyrimidinecarbaldehyde represented by the following general formula (6): 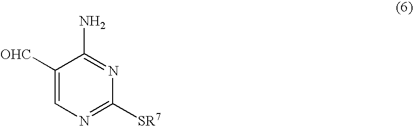
wherein R⁷ represents an alkyl group,
the process comprising reacting an alkali metal salt of 4-amino-2-mercapto-5-pyrimidinecarbaldehyde represented by the following general formula (5): 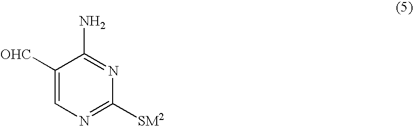
wherein M² represents an alkali metal atom
with an alkylating agent.

11. An use of an alkali metal salt of 4-amino-2-mercapto-5-pyrimidinecarbaldehyde represented by the following general formula (5): 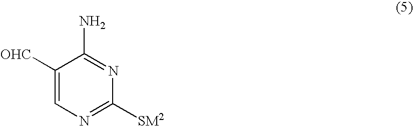
wherein M² represents an alkali metal atom
in the preparation of a 4-amino-2-alkylthio-5-pyrimidinecarbaldehyde represented by the following general formula (6): 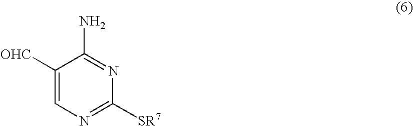
wherein R⁷ represents an alkyl group.

12. The process for preparing an alkali metal salt of 3,3-dialkoxy-2-hydroxymethylenepropanenitrile according to claim 2, wherein the base comprising an alkali metal is a base comprising a sodium atom or a potassium atom.

13. The process for preparing an alkali metal salt of 3,3-dialkoxy-2-hydroxymethylenepropanenitrile according to claim 2, wherein R⁴ is an ethyl group.

14. The process for preparing an alkali metal salt of 3,3-dialkoxy-2-hydroxymethylenepropanenitrile according to claim 3, wherein R⁴ is an ethyl group.

15. The process for preparing an alkali metal salt of 3,3-dialkoxy-2-hydroxymethylenepropanenitrile according to claim 12, wherein R⁴ is an ethyl group.

16. The process for preparing an alkali metal salt of 4-amino-2-mercapto-5-pyrimidinecarbaldehyde according to claim 8, further comprising a step for obtaining an alkali metal salt of 3,3-dialkoxy-2-hydroxymethylenepropanenitrile by the process.
